(19) United States
(12) Patent Application Publication
(10) Pub. No.: US 2024/0016402 A1
(43) Pub. Date: Jan. 18, 2024

(54) EAR-WEARABLE PHYSIOLOGY MONITORING DEVICE FOR LONG TERM COMFORTABLE WEARING AND A METHOD FOR INCREASING DIVERSITY OF MOVEMENT ARTEFACTS IN THE SIGNAL NOISE THEREIN

(71) Applicant: Ming Yip Wallace WONG, Pak Shek Kok, New Territories (HK)

(72) Inventor: Ming Yip Wallace WONG, Pak Shek Kok, New Territories (HK)

(21) Appl. No.: 17/982,523

(22) PCT Filed: Mar. 24, 2021
(86) PCT No.: PCT/CN2021/082594

§ 371 Date: Nov. 8, 2022

(30) Foreign Application Priority Data
May 7, 2020 (HK) 32020007053.2

(51) Int. Cl. A61B 5/024; A61B 5/00; A61B 5/0205

(52) U.S. Cl. CPC A61B 5/02433; A61B 5/6817; A61B 5/0205; A61B 5/7214; A61B 2562/0238

(57) ABSTRACT

An earbud comprises at least two sensing devices, each located on a different part of the earbud. The earbud is shaped or dimensioned in such a way as to instill different extents of sensing devices displacements in response to user movements.

### FIELD OF INVENTION

This invention relates to the field of wearable physiological monitors. Particularly, this invention relates to ear wearable physiological monitors.

### BACKGROUND OF INVENTION

Ear devices that contain light based physiological sensors are available for long term wearing in order to apply photoplethysmogram (PPG) technology to monitor the heart rate of the wear, i.e. the user.

However, these devices are often inaccurate when affected by user movements that add signal noise to PPG data signals. Conventionally, these earbud devices are made to fit as tightly as possible in the user's ear hole or ear canal in hope to reduce such user-movement-induced noise. Unfortunately, the tightness in the ear hole may cause discomfort after a prolonged period of wearing.

Accordingly, it is desirable to devise a method or design of an earbud device which is more comfortable to wear while having less of such movement-induced signal noise.

### SUMMARY OF THE INVENTION

In a first aspect, the invention proposes an ear-wearable physiology monitoring device, comprising: at least one emitter and at least one optical sensor in suitable numbers to provide a first emitter-to-sensor light transmission path through ear tissue and a second emitter-to-sensor light transmission path through ear tissue; the first emitter-to-sensor light transmission path being spaced apart on the device from the second emitter-to-sensor light transmission path; wherein the spacing provides that user movement causes a displacement of the first emitter-to-sensor light transmission path that is different from a displacement of the second emitter-to-sensor light transmission path.

This provides the possibility of increasing diversity of movement artefacts in signal noise caused by the same user movement, which is useful for eradication of signal noise by subsequent signal processing.

Preferably, the ear-wearable physiology monitoring device also includes an earbud; the first emitter-to-sensor light transmission path generally located on a first side of the earbud; and the second emitter-to-sensor light transmission path generally located on a second side of the earbud; wherein the first side of the earbud is a distance apart from the second side to define the spacing.

Typically, the ear bud is inserted into the ear hole. However, the skilled reader should note that “ear hole” herein refers to the actual ear canal which leads to the inner ear, as well as the surrounding areas around the mouth of the ear canal. For example, the first emitter-to-sensor light transmission path may extend through the ear tragus or helicis crus.

More preferably, the earbud has an elliptical shape when viewed axially, the elliptical shape having two relatively sharper ends and two relatively gentler sides; the first emitter-to-sensor light transmission path arranged about one of the relatively sharper ends of the elliptical shape; and the second emitter-to-sensor light transmission path arranged on one of the relatively gentler sides of the elliptical shape.

The elliptically shaped earbud provides a greater possibility that the light transmission paths are affected by user movements differently, as each different part of the elliptically shaped earbud is likely to have a different movement tendency in response to the same user movement.

Alternatively, it is possible that the first emitter-to-sensor light transmission path is arranged about one of the relatively sharper ends of the elliptical shape; and the second emitter-to-sensor light transmission path arranged about the other one of the relatively sharper ends of the elliptical shape.

Typically, the relatively sharper ends of the elliptical shape comprises a first end and a second end of the elliptical shape; the first end of the elliptical shape being sharper than the second end of the elliptical shape; the first end being capable of moving about the second end when the second end is adjacent the floor of an ear hole, wherein the first emitter-to-sensor light transmission path is arranged about the second end.

This feature provides the possibility of an egg-shaped earbud having a wider end, about which the other, sharper, end may move or wobble.

The room provided for movements or wobbling of the ear-wearable physiology monitoring device allows the user to wear the device for an extended period of time with less discomfort than a tight-fitting, not movable device.

Preferably, the ear-wearable physiology monitoring device comprises an emitter and the at least two optical sensors providing the first emitter-to-sensor light transmission path and the second emitter-to-sensor light transmission path; wherein the first emitter-to-sensor light transmission path comprises one of the at least two sensors.

Typically, the at least two sensors are placed in different locations along the axis of the earbud. This staggers the different depths in the ear hole into which the sensors are placed, creating even more diversification of movement artefacts.

Alternatively, the ear-wearable physiology monitoring device comprises an optical sensor and the at least two emitters providing the first emitter-to-sensor light transmission path and the second emitter-to-sensor light transmission path; wherein the first emitter-to-sensor light transmission path comprises one of the at least two emitters. Preferably, the at least two emitters are placed in different locations along the axis of the earbud. This staggers the different depths in the ear hole into which the emitters are placed, creating even more diversification of movement artefacts.

In a second aspect, the invention proposes a method for increasing diversity of movement artefacts in the signal noise of an ear-worn physiology monitoring device, comprising the steps of: providing a first emitter-to-sensor light transmission path through ear tissue; providing a second emitter-to-sensor light transmission path through ear tissue; wherein the two different emitter-to-sensor light transmission paths have different extents of room for displacement in response to a movement of the user.

Again, this feature provides the possibility of increasing diversity of movement artefacts in the signals obtained by the two different light transmission paths, which is useful for eradication of signal noise.

Preferably, the method further comprises a step of: locating the first emitter-to-sensor light transmission path further from a point of rotation; and locating the second emitter-to-sensor light transmission path nearer to the point of rotation; such that in response to the user movement, the first emitter-to-sensor light transmission path is capable of moving about the point of rotation over a greater distance than the second emitter-to-sensor light transmission path according to the different extents of room for displacement.

The skilled reader would understand that the point of rotation need not be a physical point but merely a mathematically definable point.

Optionally, the method includes a step of: performing a linear combination with a pre-defined ratio imposed on signals obtained from the two different emitter-to-sensor light transmission paths to remove movement artefacts. This feature helps to eradicate the noise components that are induced by user movements.

In a further aspect, the invention provides an ear-wearable physiology monitoring device, comprising: at least one emitter and at least one optical sensor in suitable numbers to provide a first emitter-to-sensor light transmission path through ear tissue and a second emitter-to-sensor light transmission path through ear tissue; the first emitter-to-sensor light transmission path being spaced apart on the device from the second emitter-to-sensor light transmission path; wherein the spacing provides that user movement causes a displacement of the first emitter-to-sensor light transmission path and a displacement of the second emitter-to-sensor light transmission path that is about a pivotal point distal from the axis of the ear hole.

Typically, the “pivotal point” is a mathematical pivot that is distal from the axis of the ear hole. The pivotal point is not aligned to the axis of the ear hole, such that the ear-wearable physiology monitoring device does not rotate about itself and the axis of the ear hole. Even if the earbud is completely round in shape, this provides an advantage that the first emitter-to-sensor light transmission path and the second emitter-to-sensor light transmission path experience different extent of movements, which further diversifies the movement artefacts in signal noise. In contrast, if the first emitter-to-sensor light transmission path and the second emitter-to-sensor light transmission path rotate about a central axis that is aligned to the axis of the ear hole then, unless the earbud is not round in shape and having the central axis as the point of origin and the light transmission paths are placed at different radial distance from the axis, the light transmission paths may well experience very similar moment artefacts.

In yet a further aspect, the invention proposes an ear-wearable physiology monitoring device, comprising: at least one emitter and at least one optical sensor in suitable numbers to provide a first emitter-to-sensor light transmission path through ear tissue and a second emitter-to-sensor light transmission path through ear tissue; at least one emitter and at least one optical sensor placed on independently movable surfaces on the device; wherein the independently movable surfaces provides that user movement causes a displacement of the first emitter-to-sensor light transmission path that is different from a displacement of the second emitter-to-sensor light transmission path.

### BRIEF DESCRIPTION OF THE DRAWINGS

It will be convenient to further describe the present invention with respect to the accompanying drawings that illustrate possible arrangements of the invention, in which like integers refer to like parts. Other arrangements of the invention are possible, and consequently the particularity of the accompanying drawings is not to be understood as superseding the generality of the preceding description of the invention.

FIG. 1 is a drawing of a human ear on which an embodiment such as that in FIG. 2 may be worn;

FIG. 2 shows the axial cross-sectional view of an embodiment of the invention;

FIG. 3 shows how the embodiment of FIG. 2 is able to obtain user physiological information;

FIG. 3a shows the pulse of a user that may be obtained using the embodiment of FIG. 2;

FIG. 3b schematically shows how the pulse FIG. 3a may be obscured by signal noise induced by user movements;

FIG. 4 shows how the embodiment of FIG. 2 is able to increase diversity of movement artefacts;

FIG. 4a is a comparative example to FIG. 4;

FIG. 4b schematically illustrates some types of different phase lags between sensors of different embodiments;

FIG. 4ba illustrates a simple embodiment which is alternative to the embodiment of FIG. 4;

FIG. 4c illustrates the pulse of a user of the embodiments;

FIG. 4d illustrates signals caused by movements of a user of the embodiments;

FIG. 4e illustrates the output of a sensor in the embodiments which combines the signals of FIG. 4c and FIG. 4d;

FIG. 4f illustrates how the movement signals in the output of a transmission path can be out of phase with the movement signals in the output of another transmission path;

FIG. 5 is a photograph of a prototype which may comprise the embodiment of FIG. 2;

FIG. 6 is a view of the prototype of FIG. 5 from one direction;

FIG. 7 is a view of the prototype of FIG. 5 from another direction;

FIG. 8 shows a variation of the embodiment of FIG. 11;

FIG. 9 shows how the embodiment of FIG. 8 is worn in the ear

FIG. 10 shows a prototype that comprises the embodiment of FIG. 8;

FIG. 11 shows the axial cross-sectional view of a second embodiment;

FIG. 12 shows how the embodiment of FIG. 11 is able to obtain user physiological information;

FIG. 13 is a perspective view of the embodiment of FIG. 11;

FIG. 14 is a perspective view of the embodiment of FIG. 11;

FIG. 15 shows how the embodiment of FIG. 11 provides redundancy in the collected signals;

FIG. 16 shows how the embodiment of FIG. 11 is worn in the ear:

FIG. 17 shows how the embodiment of FIG. 11 is able to increase diversity of movement artefacts;

FIG. 18 shows a variation of the embodiment of FIG. 11:

FIG. 19 shows a variation of the embodiment of FIG. 5:

FIG. 20 shows the light transmission pathways of the embodiment of FIG. 19; and

FIG. 21 shows another embodiment.

### DESCRIPTION OF EMBODIMENTS

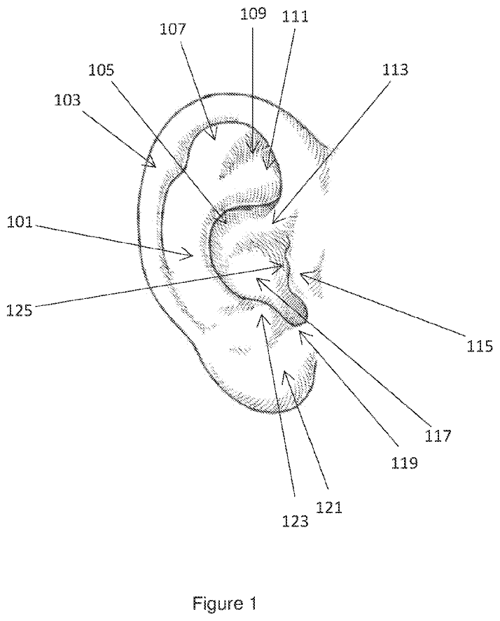

FIG. 1 shows the anatomy of the outer ear, in which one finds the portions named anti-helix 101, helix 103, concha cymba 105, superior crus 107, triangular fossa 109, inferior crus 111, helicis crus 113, tragus 115, concha cavum (usually known as concha for short) 117, intertragic notch 119, lobule 121, anti-tragus 123. The tragus 115 is a small pointed eminence of the external ear in front of the concha 117, and projecting backwardly over the ear hole or ear canal. The nearby anti-tragus 123 projects forwardly and upwardly.

The opening to or mouth of the ear hole 125, is not visible in the drawing but the skilled reader would understand that the ear hole 125 is typically right behind the tragus and extends into the inner ear.

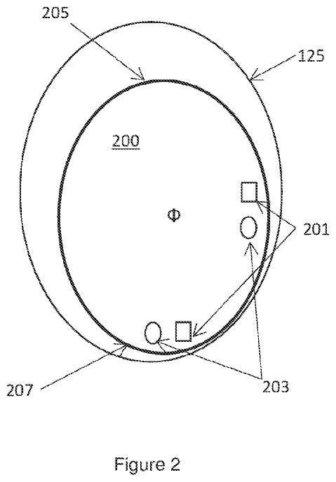

FIG. 2 is a schematic view of the axial cross-section of an earbud 200. That is, the view in FIG. 2 is aligned to the ear hole 125 when the earbud 200 is inserted therein. The centre or the axis of the cross-section is marked with ‘ϕ’. The cross-sectional area and shape of the earbud 200 is smaller than the cross-sectional area and shape of the ear hole 125 in order to allow some movements of the earbud 200 within the ear hole 125. Preferably, the cross-sectional shape is elliptical or oval. An elliptical shape has two sharper ends, which are more curved than the sides connecting the two ends. In other words, the sides have a gentler curvature then the ends of the elliptical shape.

Since there is room for earbud movements, the earbud does not plug the ear hole so tightly, and this allows the earbud to be worn for an extended period of time.

The earbud 200 is provided with light emitters (emitters 201) and two optical sensors (sensors 203) which may be grouped as two pairs of PPG detectors, and which provide four emitter-to-sensor light transmission paths. The rectangles in FIG. 2 represent the emitters 201 while the circles represent the sensors 203. The meaning of “pair” here is illusory, to mean that the emitter 201 and sensor 203 of each pair may be placed closer to each other than to the emitter 201 and sensor 203 of the other pair. However, as each of both sensors 203 is capable of detecting light from both the emitters 201, this ensures that the light from either one of the emitters 201 reaches each one of the sensors 203 from different distances, different angles and different light transmission paths.

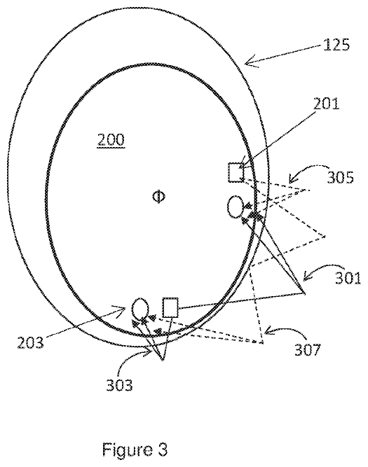

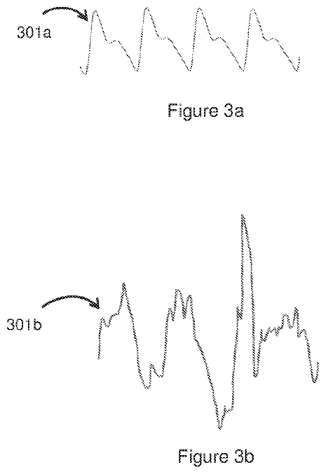

FIG. 3 shows how light travel through different light transmission paths 301, 303, 305, 307, angles and directions from any one of the emitters 201 is able to reach both the sensors 203. Light, shown in dashed lines, emitted from one of the emitters 201 penetrates the skin of the ear hole 125 and travels within the user's ear tissue.

Typically, the emitters 201 emit light in any pre-selected wavelength which can be absorbed by blood in the tissue of the ear hole 125. Correspondingly, the sensors 203 are able to detect the specific wavelength of light emitted by the emitters 201. Light emitted by the emitters 201 penetrates through the skin and tissue of the ear hole 125. Some of the light is absorbed by blood in the tissue and is converted into heat or other form of energy but some other of the light is simply reflected internally in all directions within the tissue. As a result, light is simply scattered within the tissue. Some of the scattered light exits from the tissue back into the ear hole 125 and reaches one of the sensors 203 by one light transmission path 301, while some other of the scattered light reaches the other one of the sensors 203 by another light transmission path 303.

The light transmission paths 305, 307 of light from the other emitter 201 reaches the two sensors 203 in the same way and are shown in solid lines.

Light reaching the sensors 203 through such light transmission paths 301, 303, 305, 307 has a pulsating intensity which is due to the pulsating volume of blood in the tissue. Consequently, the amount of light which passes through the tissue to reach the sensors 203 is observed by the sensors 203 to manifest fluctuations. By suitable signal analysis, the pulse of the user can be observed thereby to deduce his heart condition, blood pressure, fitness and exercise effectiveness, and even psychological stress level.

Preferably, the emitters 201 take turn to emit light, and this allows both sensors 203 to pick up the signals from the same emitter 201 at any point in time. For completeness, it is mention now that the frequency at which the emitters 201 switch over each to the other is very fast, and is often many times within the short period of a pulse.

Optionally, the emitters 201 are each overlaid with an optical filter (not illustrated) to permit emission of a different wavelength. This helps to observe the user for physiological data using different wavelengths.

Alternatively, the sensors 203 are each overlaid with an optical filter (not illustrated) to permit passage of a different wavelength. In this case, the selected wavelength is typically within the emission spectra of both the emitters 201.

In a variation of the embodiment, both emitters 201 and sensors 203 may be switched on all the time, since the detection of both the light emitted from the two emitters 201 by both the sensors 203 has an effect of signal addition which can eliminate noise.

The light transmission paths to each of the sensors 203 from both the emitters 201 are preferably as different as possible, e.g. from different directions or from different angles, to provide diversity in signal noise. Having more diversity in signal noise allows the noise to be treated in order to expose the pulse of the user more prominently. However, to provide even greater diversity in the signal noise, the embodiment provides the possibility that the different light transmission paths 301, 303, 305, 307 are affected differently by any of the user's movements, that is, by situating the emitters 201 and sensors 203 defining the light transmission paths 301, 303, 305, 307 in different parts of the earbud 200 that are have different extents of physical displacements in response to a same user movement.

Typically, user movements jerk the earbud 200 and cause erratic signal noises which overlays the desired physiological data signals. These movement induced signal noise mars the reading of the data signals. FIG. 3a shows an example of a user's pulse signal 301a that may be observed by using the emitters and sensors. FIG. 3b shows the readings of the emitters and sensors being overwhelmed by signal noise 301b that have magnitudes so big that the pulse signal is completely hidden. Typically, signal noise of such magnitudes is due to movements of the user.

The prior art tried to reduce movement-induced signal noise by making the earbud 200 so big that the earbud 200 plugs the ear hole 125 as tightly as possible, and so that the earbud 200 does not move inside the ear hole 125. In opposite teaching to the prior art, the embodiment increases diversity in signal noise by allowing different extents of room for movements to different parts of the earbud 200. This provides that movement-induced signal noise may be more easily treated and removed by known digital signal processing methods. Therefore, signal noise may be treated to allow the embodiment to provide more accurate pulse and physiological data despite user movements which would have otherwise obscured the data signals.

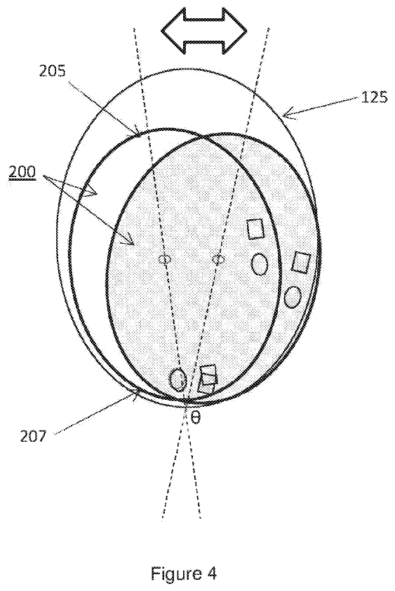

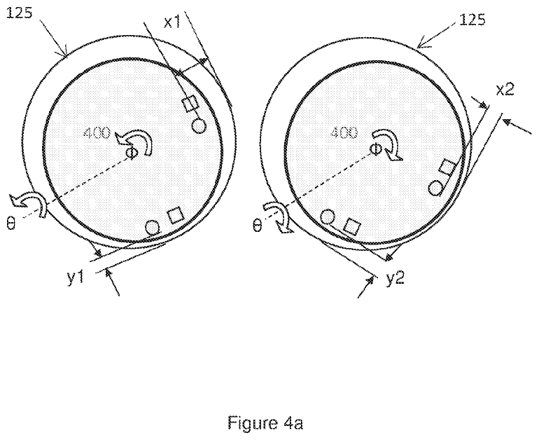

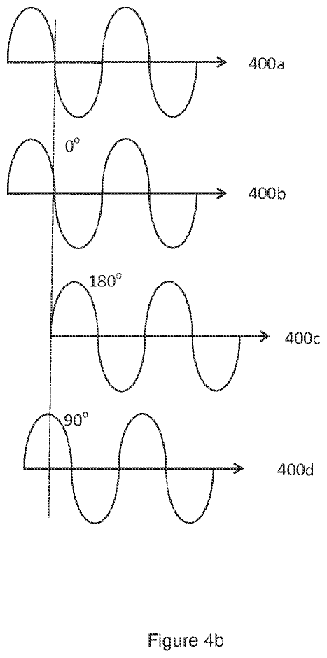

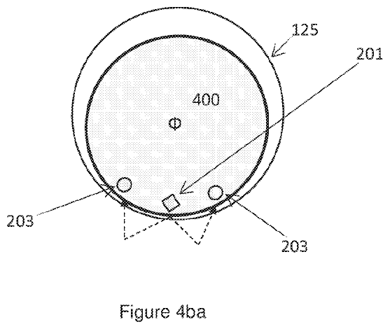

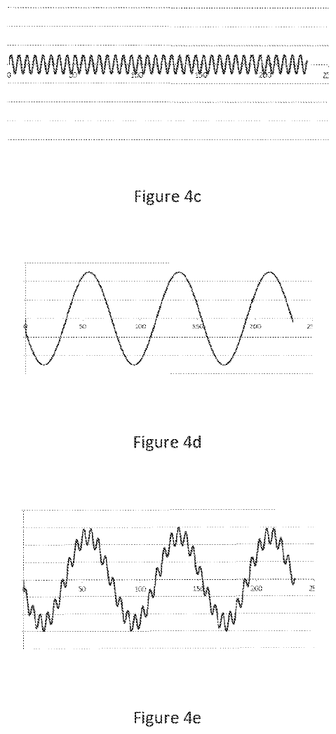

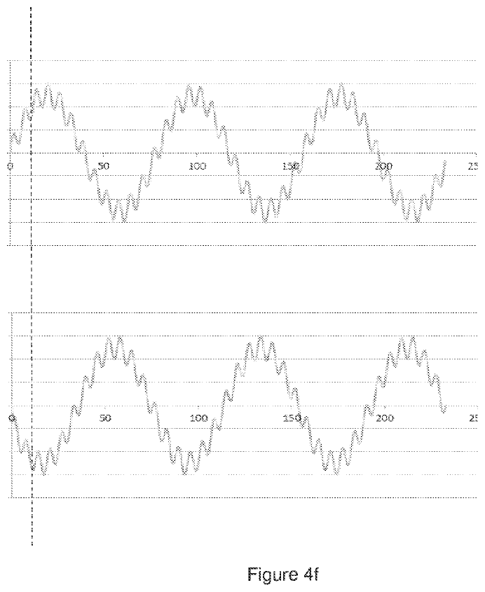

For example, due to the pull of gravity, as illustrated in FIG. 4, the bottom end 207 of the earbud 200 is likely to be in contact with the floor of the ear hole 125 and is more stable, or less prone to being displaced than the top end 205 of the earbud 200. When the user wearing the earbud 200 makes sudden or big movements, the earbud 200 moves in the ear hole 125 too. However, the bottom end 207 of the earbud 200 move to a lesser extent as it is adjacent, and is probably in contact with, the floor of the ear hole 125. In contrast, the freer top end 205 of the ear bud is likely to move about or wobble more, and generally moving about the relatively more stable bottom end 207. This is illustrated as displacement of the embodiment about a pivotal point 9 that is preferably eccentric or distal to the axis of the ear hole 125, ϕ. As the skilled man can see, the pivotal point θ is not a physical pivot but a mathematical one. This provides that the light transmission path nearer the top end 205 of the earbud 200 gets angularly displaced over a greater possible extent than the light transmission path nearer the bottom end 207 of the earbud 200.

During the wobbling of the top end 205, the emitters 201 emit into different parts of the ear hole 125 wall, and the sensors 203 detect light that has travelled through continuously changing light transmission paths through ear hole 125 tissue. This diversifies and randomises the light transmission paths. The skilled reader would understand that a single pulse of the user may be constructed of several readings over a few quick wobbles.

FIG. 4a is a comparative embodiment which is less effective. A similar earbud 400 as in FIG. 4 is shown in FIG. 4a, except that the earbud 400 is round and the pivotal point θ is aligned to the axis of the earbud 400 and also to the axis of the ear hole 125. That is, the earbud 400 of FIG. 4a may rotate about its actual axis ϕ instead of a distal pivotal point, as illustrated by the white arrows. The left drawing in FIG. 4a shows a rotation to the left about aligned axes θ and ϕ. The right drawing in FIG. 4a shows a rotation to the right about aligned axes θ and ϕ. In whichever direction the earbud in FIG. 4a rotates, the respective distances between both sensors to ear tissue reduce or increase roughly the same, resulting in the sensors signals being affected by a user movement in the same or highly similar way, i.e. the movement artefacts are in phase and has similar magnitudes. In the arrangement shown in FIG. 4a, if the earbud of FIG. 4a rotates from the left to the right, the distance between one sensor from the ear tissue x1 become a smaller x2, while the distance between the other sensor and the respective part of the ear tissue y1 becomes a greater y2 at the same time, resulting in correspondingly out-of-phase movement artefacts.

Mathematically, treatment of the signal noise may be explained as follows.

S1(n)h)+m1(n) (1)

S2(n)=h2(n)+m2(n) (2)
Where
Sx(n) is the overall signal from sensor x, and
hx(n) is signal output due to the pulse of the user for sensor x, and
mx(n) is motion noise caused by user movements in sensor x.

A program or firmware in a microprocessor (not illustrated) contained in the earbud (or even an external processor as the case may be) adjusts the intensity of light emitted by the emitters until ∥h1(n)∥=∥h2(n)|, in which case h1(n) and h2(n) becomes in-phase for the same movements, i.e. frequency of movements as a signal.

In most situations,

$$\frac{‖ h 1 ( n ) ‖}{‖ m 1 ( n ) ‖} \neq \frac{‖ h 2 ( n ) ‖}{‖ m 2 ( n ) ‖}$$

as the ear hole location of sensing is different for the different sensors, and the density of blood vessels in ear tissue in the sensor location is different, and also the distance between each sensor and the ear hole wall is likely to be different.

However, where m1(n) and m2(n) are caused by the same user movements, it is possible to the sensor outputs are either in phase or 180 degrees out of phase, or anywhere in between, depending on the location of the sensors and/or the emitters.

As ∥m1(n)∥>>∥h1(n)∥, hence ∥Sn(n)∥=∥m1(n)∥.

Assuming that m1(n) and m2(n) are out of phase, the following may be derived.

$$Sr ( n ) = S 1 ( n ) \times ‖ m 2 ( n ) ‖ + S 2 ( n ) \times ‖ m 1 ( n ) ‖ = h 1 ( n ) \times ‖ m 2 ( n ) ‖ + m 1 ( n ) \times ‖ m 2 ( n ) ‖ + h 2 ( n ) \times ‖ m 1 ( n ) ‖ + m 2 ( n ) \times ‖ m 1 ( n ) ‖$$

In the case where m1(n) and m2(n) are in phase, m1(n)×∥m2(n)∥=−m2(n)×∥m1(n)∥. That is, as m1(n) and m2(n) are in-phase, the “+” signal may be replaced with “−”. The calculation still applies but it will be less effective as the part of the sensor signal will be cancelling each other. Accordingly, out-of-phase movement artefacts are preferred.

Hence Sr(n)=h1(n)×∥m2(n)∥+h2(n)×∥m1(n)∥=k h(n), where k is a constant.

The number of signals Sx(n) can be expanded to a larger number x, to compensate for any error introduced by m1(n), m2(n) that are not completely in phase or out of phase.

FIG. 4b illustrates what it means to be in or out of phase. The top graph 400a in FIG. 4b represents movements of the user (not his pulse) as read by a light transmission path. The bottom three graphs 400b, 400c, 400d represent three possible readings by a second light transmission path. If two light transmission paths in the embodiment are varied to different extents by the same user movements, the detection of the user movements as observed by the different light transmission paths may have the same movement signal shape (or user movement frequency) but there may be a lag in the readings of the different sensors. If the readings of both the sensors are completely in phase and there is no lag, i.e. at zero degree, the top graph 400a and the second graph 400b will manifest from the two light transmission paths. If the readings of both the sensors are completely out of phase or lagging by 180 degrees, the top graph 400a and the third graph 400c will manifest from the two light transmission paths. If the readings of both the sensors are lagging by 90 degrees, the top graph 400a and the fourth graph 400d will manifest from the two light transmission paths.

FIG. 4ba shows an embodiment that may provide a completely out of phase lag in two light transmission path readings. In the embodiment of FIG. 4ba, there is one emitter 201 placed at the bottom of the ear bud. Two sensors 203 are provided, each of which is placed on either side of the emitter. Together, the one emitter and two sensors provide two light transmission paths. If the embodiment of FIG. 4ba rolls to the right of the drawing, the sensor on the right moves closer to ear tissue while the sensor on the left moves away from ear tissue. In this case, the sensor on the right is affected by a movement artefact and the sensor to the left is affected by the same movement artefact but in a negative way. This will create a 180 degrees lag between the part of signal noise caused by user movement, i.e. the top graph 400a for one sensor and third graph 400c of FIG. 4a for the other sensor.

Going back to FIG. 4a, in that embodiment, the movement signals between the two sensors will be 90 degree out of phase as the sensors are not placed on symmetric locations about a pivotal point, i.e. one sensor is nearer the floor of the earhole while the other is nearer the side of the earhole. In cases like this where signal noise caused by movements are not 180 degree out of phase, at least data from three light transmission paths will be needed to remove the movement artefacts mathematically.

As mentioned, however, the embodiment of FIG. 4a is not preferred. This is because the cross-sectional shape the earbud is round, and the pivotal point of rotation is central to the round as well as aligned to the centre of the ear hole or ear canal, as there is less diversification of the movements artefacts. In general, to improve diversity in movement artefacts, embodiments that have a round cross-sectional shape preferably have a pivotal point of turning that is eccentric or distal from axis of the ear hole or ear canal, and embodiments that have a pivotal point of turning that is aligned to the axis of the ear hole or ear canal preferably have a non-round cross-sectional shape.

FIG. 4c, FIG. 4d, FIG. 4e and FIG. 4f illustrate the afore-described mathematical treatment graphically, and show why the pulse and movement signals may be separated. FIG. 4c represents the pulse of the user. FIG. 4d represents a large noise signal caused by movements of the user. FIG. 4e is the combined signal which is observed by one of the sensors. The purpose of the embodiment is to separate the mixed signal in FIG. 4e to retrieve the pulse signal in FIG. 4c.

Magnitude and frequency of the signals used in the example are exaggerated for illustrative purposes, as the skilled man ought to know that movement frequency may well be faster than pulse signal in many circumstances, such as when the user is animated in a social even but remain emotionally calm and not engaging in physical exercise.

FIG. 4f shows the readings or output of two light transmission paths (from two sensors or, alternatively, the output of one sensor that quickly and alternately samples light transmitted from two emitters). If the two sensors are arranged in the configuration of FIG. 4ba, where when one sensor moves towards the ear tissue, the other sensor correspondingly moves away from the ear tissue in the opposition direction, the movement signals of the two sensors would be completely out of phase by 180 degrees. However, pulse signals of the user as observed by both sensors do not lag behind one after the other, the pulse as observed by the sensors is always the same and in phase. FIG. 4f also shows how the pulse is in phase by the vertical dashed line. Hence, the 180 degrees out of phase movement signals picked up by the two sensors, which are signal noise due to use movements, may be added to cancel out each other, but the pulse signals which are in phase do not cancel out. In this way, the pulse of FIG. 4c may be retrieved. More complicated signal processing techniques or mathematical treatment may be applied when there are more than two sensor signals in various degrees of lag, but these are downstream to the embodiment and do not form part of the invention. For example, the raw signal output of the different sensors 203 may be added together by a process of linear combination with a pre-defined ratio or weightage imposed on the signals of the different sensors 203. Nevertheless, it suffices here to illuminate the skilled man with this most simple example.

Accordingly, as illustrated by the foregoing embodiments, to increase the likelihood of out-of-phase movement artefacts, diversification of the movement artefacts may be increased by placing some of the emitters 201 and sensors 203 nearer the top end 205 and the other of the emitters 201 and sensors 203 nearer the bottom end 207, so that the emitters 201 and sensors 203 in these different locations are moved to different extents despite being cause by the same user movement. In other words, subjecting different ones of the emitters 201 and sensors 203 to different movements diversifies the movement artefacts in their signal output. Such diversified movement-induced signal noise can be used to cancel out each other so that the underlying physiological signal may be manifested more easily. This allows the earbud 200 to become more robust and stable in use, which is particularly desirable to a user who wishes to have his physiological data monitored while he is engaging in a strenuous physical exercise.

As the skilled man knows, signal diversity is not the same as signal randomness. Randomness refers to the characteristics of white noise, i.e. white noise that exists in all wavelengths and is not removable by signal processing techniques.

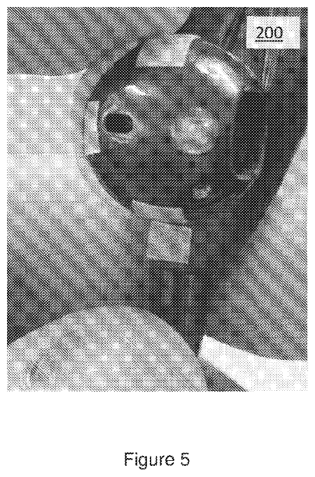

FIG. 5 is a photograph of a prototype of an embodiment, which is the earbud 200 of a portable earphone. The same reference numeral ‘200’ is used to indicate the earbud in FIG. 2 and in FIG. 5 as they are like parts. The earbud 200 may contain necessary electrical and optical components including microprocessors (not visible) to operate as an ear-wearable physiology monitoring device. Generally, ‘earbud 200’ refers to the part of the earphone which inserts, whether fully or partially, into the ear hole 125. Although an earphone is shown, the skilled reader would understand that other earbuds having non-earphone functions are within the contemplation of this description, and these include even ear studs for blocking water from entering the ears of swimmers that also have swimmer physiology monitoring functions.

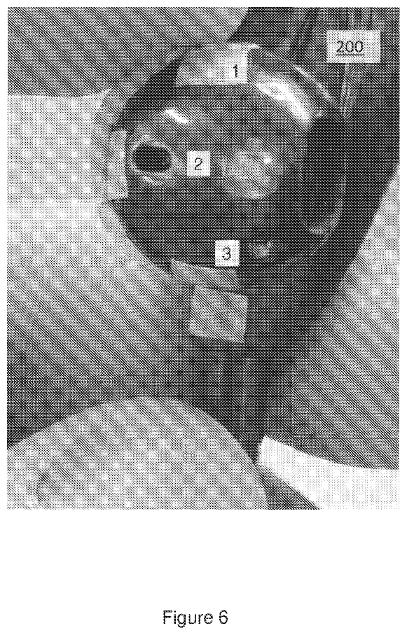

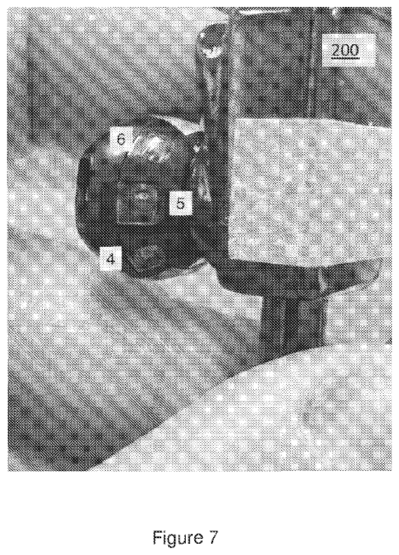

FIG. 6 and FIG. 7 corresponds to the photograph in FIG. 5, and show the same prototype from opposite directions. There are emitters and sensors arranged on the sides of the earbud 200 to provide physiology monitoring of the user using, though not necessarily, techniques such as photoplethysmogram (PPG). The labelled portions 1, 3 and 5 are LEDs, which is a preferred type of emitters. The portions labelled 2, 4 and 6 are the sensors. Light emitted by any of the emitters may penetrate the wall of the ear hole 125 to enter into the tissues defining the ear hole 125, and then exit from the tissues back into the ear hole 125, to be picked up by any one of the sensors.

In one of the simplest embodiments, however, the LEDs labelled with numerals 3 and 5, and the sensor labelled with numeral 6 in FIG. 6 and FIG. 7 are not provided and would be absent therefrom. This provides a one emitter to two sensors configuration, which are sufficient to provide at least two emitter-to-sensor light transmission paths. The two emitter-to-sensor light transmission paths are would be moved to different extents when the earbud 200 is rotated, tilted or wobbled when worn in the ear hole 125 of a user.

In another one of the simplest embodiments, the LED labelled with numeral 5, and the sensors labelled with numerals 4 and 6 in FIG. 6 and FIG. 7 are not provided and would be absent therefrom. This provides a one sensor to two emitters configuration, which are also sufficient to provide at least two emitter-to-sensor light transmission paths. Again, the two emitter-to-sensor light transmission paths would be moved to different extents when the earbud 200 is rotated, tilted or wobbled when wom in the ear hole 125 of a user.

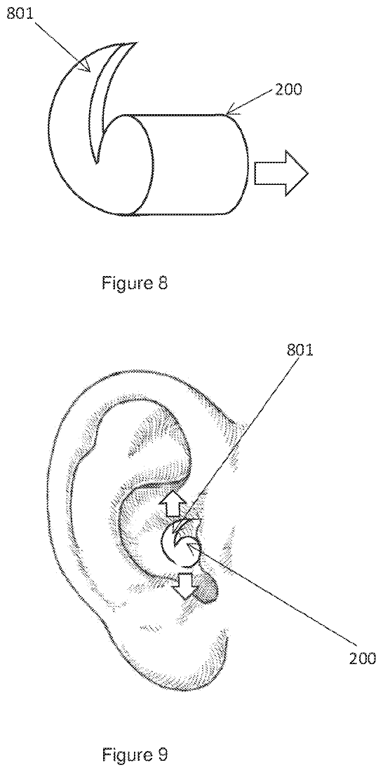

Generally, it is rather difficult to secure an earbud 200 into the ear hole 125 if the earbud 200 has a size that does not fill up the entire ear hole 125. Hence, variations of the embodiments that may secure the undersized earbud 200 is within the contemplation of this description. For example, as shown in FIG. 8, an arm 801 made of tough but resilient plastic material such as high density polyethylene or silicone may extend from the side of the earbud 200 that faces away from the user when wom. The arrow in FIG. 8 shows the direction of insertion into the ear hole 125. The white arrows in FIG. 9 illustrate how the arm 801 maybe placed to exert an upward biasing force against the underside of the helicis crus 113. The earbud 200 is illustrated a little bigger than actual size for clarity. The biasing force allows the top end of the earbud 200 to move more freely by pushing down the bottom end 207 into gentle contact with the floor of the ear hole.

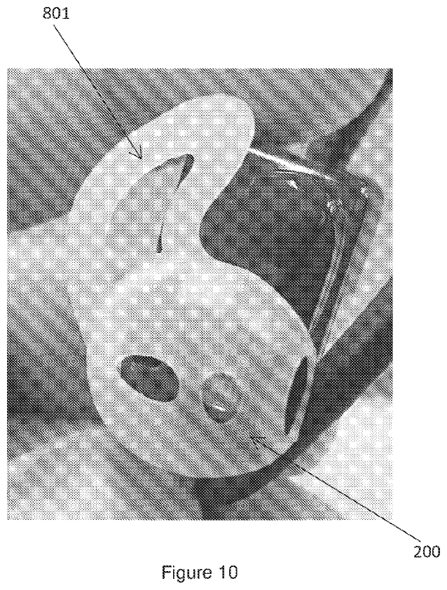

FIG. 10 is a photograph of a prototype that has a similar arm 801.

Alternatively, instead of the arm 801 providing support to the earbud 200 for securing the earbud 200's position in the ear, the earbud 200 can be wrapped with a layer of very transparent and soft material such as silicone (not illustrated). The layer of silicone is inserted into and fills the ear hole 125, with the earbud 200 encapsulated inside. The softness of silicone allows positional displacement of the earbud 200 inside the ear hole 125.

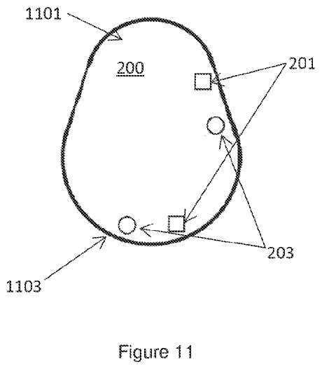

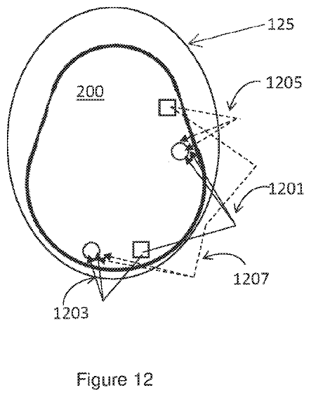

FIG. 11 shows another embodiment, in which the cross-sectional shape of the earbud 200 is not only elliptical, but also bigger at one end, which is also known as egg-shaped or pear-shaped. Hence, the bottom end 1103 of the earbud 200 being bigger fills up the floor of the ear hole 125 more than the top end 1101 of the earbud 200 filling up the space near the roof of the ear hole 125. FIG. 12 is a corresponding drawing to the embodiment of FIG. 11, showing how light from anyone of the emitters 201 is still able to reach both the sensors 203, which work in the same way as described for FIG. 3, through different transmission paths 1201, 1203, 1205, 1207. The relatively heavier bottom end 1103 lends the bottom end 1103 greater stability, ensuring the diversification of movement induced signal noise when the top end 1101 of the egg-shaped earbud 200 wobbles about the bottom end 1103.

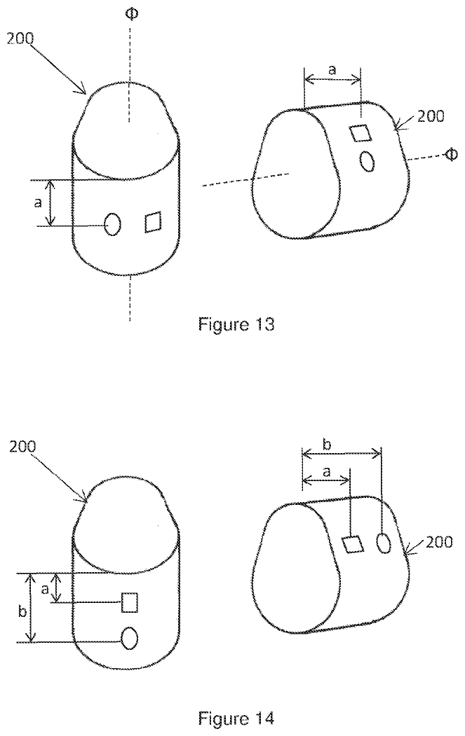

FIG. 13 and FIG. 14 each shows a perspective view corresponding to the view shown in FIG. 11. The earbud 200 is actually an extended body that can be inserted into the ear hole 125, the sensors 203 and the emitters 201 are shown placed along the length of the extended body. In FIG. 13, the sensors 203 and emitters 201 are placed to the same depth ‘a’ from the side of the earbud 200 that faces away from the user when worn. FIG. 14 shows a variation in which the sensors 203 and emitters 201 are placed to staggered depths of ‘a’ and ‘b’ from the side of the earbud 200 that faces away from the user when worn. Staggering the position along the depth of the ear hole further enhances diversification of movement artefacts resulting from user movements.

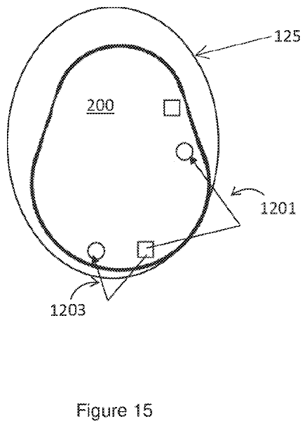

FIG. 15 illustrates a further advantage of the embodiment. If one of the two emitters 201 breaks down, the remaining emitter 201 is capable of emitting light that travels through different light transmission paths 1201, 1203 to reach the two sensors 203. This means the embodiment has a redundancy factor by one emitter 201. This adds to the lifespan of a product based on the embodiment.

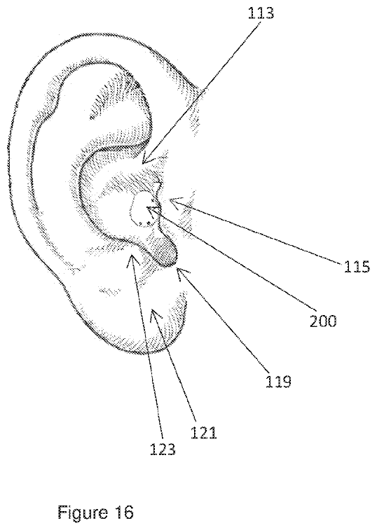

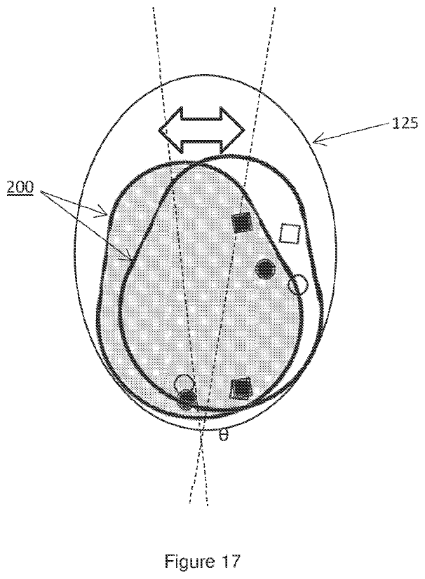

FIG. 16 shows the earbud 200 of FIG. 11 inserted into the ear hole 125 of the user. As illustrated in FIG. 17, this provides an advantage that the earbud 200 is able to wobble about the bottom end 1103, and about an eccentric or distal pivotal point θ that is not aligned to the axis of the ear hole. There are more room for displacement of the top of the earbud 200 than the bottom end 1103 of the earbud 200 as the user moves. This produces different movement artefacts in the signals obtained by the sensor 203 nearer the top of the earbud 200 than in the signals obtained by the sensor 203 nearer the bottom end 1103 of the earbud 200 even though the noise were triggered by the same user movement. In this way, the shape and size of the earbud 200 encourages one part of the earbud 200 to move more than another part of the earbud 200 in response to the same user movement. Sensors 203 may be placed on these different parts of the earbud 200 to diversify the effect of user movements on the sensor 203 signals, improving the possibility of cancelling some or all of the noise in the signals.

If a complementary second earbud 200 is worn on the other ear, it becomes even more possible for signal noise that is caused by user movements to be diversified and cancelled. The underlying pulse signal is obtained from virtually periodic changes of blood content in the blood vessels, caused by the pumping heart, and is always in phase despite being read by any number of light transmission paths. Hence, the underlying signal is the same in both ears and may be added to reduce signal noise.

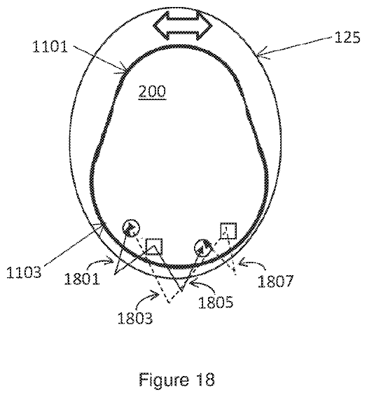

FIG. 18 shows another arrangement of the emitters 201 and sensors 203, in which the emitters 201 and sensors 203 are placed at the bottom end 1103. As explained for the embodiments afore-described, the light emitted by each emitter 201 is capable of reaching both the sensors 203, providing four different light transmission pathways 1801, 1803, 1805, 1807. While this configuration is still advantageous over the prior art, this has less diversification of the movement artefacts in signal noise caused by user movement, since all the light transmission pathways are placed about the bottom end 1103 of the embodiment.

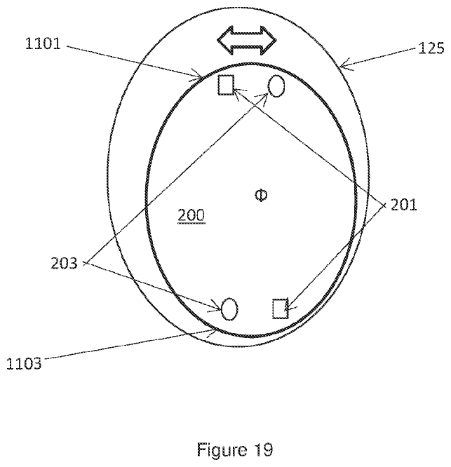

FIG. 19 shows yet another embodiment which is within the contemplation of this description, having an arrangement of the emitters 201 and sensors 203 in an elliptically shaped embodiment instead of a pear-shaped embodiment, in which one pair of the emitters 201 and sensors 203 are placed at the bottom end 1103 and another pair of the emitters 201 and sensors 203 are placed at the top end 1101.

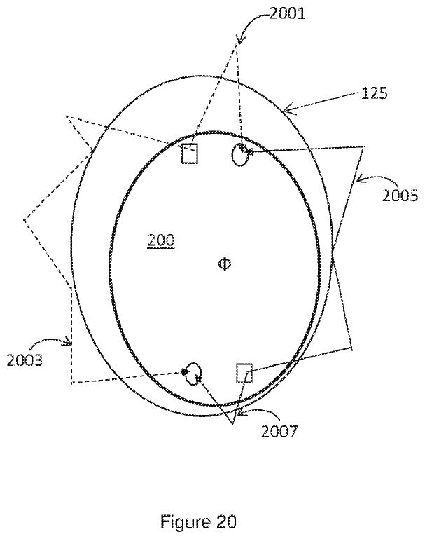

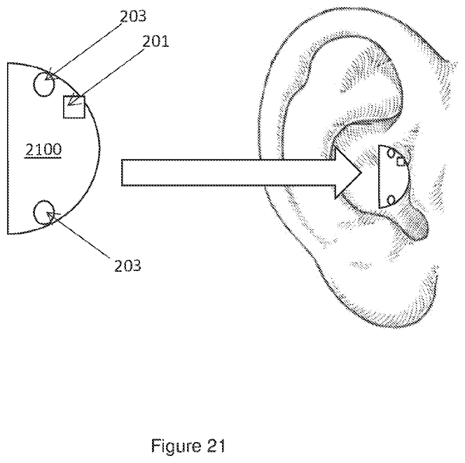

The doubled headed arrow shows illustrates the tendency of the top end 1101 to wobble about a relatively stable bottom end 1103. The light transmission pathways 2001, 2003, 2005, 2007 of this embodiment are shown in FIG. 20. As the two pairs of emitter and sensor are placed at the extreme ends of the elliptically shaped embodiment, two of the light transmission pathways 2001, 2007 are quite short in distance while the other light transmission pathways 2003, 2005 pass through greater distances in the tissue of the ear hole.

FIG. 21 show another embodiment 2100 which does not fit inside the ear hole or the ear canal. Instead, the embodiment has a curved surface that is suitable for being cradled in the concha of the ear. The provided emitter and sensors (or any other combination and number of sensors and emitters) provide at least two light transmission paths through tissues of the outer ear. Hence, the skilled reader ought to note that the meaning of ‘ear hole’ is not limited to the ear canal or the mouth of the ear canal. Some embodiments may apply the emitters and sensors onto parts of the ear concha, the tragus, helicis crus and so on, as along as suitable light transmission paths may be provided by placement of the emitters and sensors. In other words, the meaning of ear bud also applies to any configuration of a ear device which is capable of being cradled by any part of the ear to provide the light transmission pathways.

Accordingly, the embodiments include an ear-wearable physiology monitoring device, comprising: at least one emitter 201 and at least one optical sensor 203 in suitable numbers to provide a first emitter 201-to-sensor 203 light transmission path and a second emitter 201-to-sensor 203 light transmission path; the first emitter 201-to-sensor 203 light transmission path being spaced apart from the second emitter 201-to-sensor 203 light transmission path; wherein the spacing provides that user movement causes a displacement of the first emitter 201-to-sensor 203 light transmission path that is different from a displacement of the second emitter 201-to-sensor 203 light transmission path when the device is worn on the ear of a user.

Furthermore, the embodiments include a method for increasing diversity of movement artefacts in the signal noise of an ear-worn physiology monitoring device, comprising the steps of: providing a first emitter 201-to-sensor 203 light transmission path; providing a second emitter 201-to-sensor 203 light transmission path; allowing one of the two different emitter 201-to-sensor 203 light transmission paths to have a different extents of room for displacement relative to movements of the user than the other one of the two different emitter 201-to-sensor 203 light transmission paths.

While there has been described in the foregoing description preferred embodiments of the present invention, it will be understood by those skilled in the technology concerned that many variations or modifications in details of design, construction or operation may be made without departing from the scope of the present invention as claimed.

Other embodiments may include further functions such as detection of heart rate, or include a voice prompt to help the user in an exercise routine.

The skilled reader understands that the emitters 201 may be emitting in different colours or light frequencies and, in some embodiments, even invisible wavelengths.

For example, one emitter 201 may emit red light while the other in infra-red, both emitters 201 may emit in different infra-red wavelengths, both emitters 201 may emit in the same infra-red wavelength, one emitter 201 may emit light in ultraviolet while the other emitter 201 emit in infra-red, one emitter 201 may emit light in a green light while the other emitter 201 emit in red light. The ways to ensure the sensors 203 detect different wavelengths include staggering the operations of the emitters 201 or staggering the operations of the sensors 203, staggering the operations of the emitters 201.

Although the embodiments described uses PPG for detecting blood volume changes in the microvascular bed of tissue, the optical sensors 203 may be used in other optical techniques to detect other physiological information, such as detection of blood glucose, oxygen level, hydration level and so on.

1. An ear-wearable physiology monitoring device, comprising:
at least one emitter and at least one optical sensor in suitable numbers to provide a first emitter-to-sensor light transmission path through ear tissue and a second emitter-to-sensor light transmission path through ear tissue;
the first emitter-to-sensor light transmission path being spaced apart on the device from the second emitter-to-sensor light transmission path; wherein
the spacing provides that user movement causes a displacement of the first emitter-to-sensor light transmission path that is different from a displacement of the second emitter-to-sensor light transmission path.

2. An ear-wearable physiology monitoring device as claimed in claim 1, further comprising:
an earbud;
the first emitter-to-sensor light transmission path generally located on a first side of the earbud; and
the second emitter-to-sensor light transmission path generally located on a second side of the earbud; wherein
the first side of the earbud is a distance apart from the second side to define the spacing.

3. An ear-wearable physiology monitoring device as claimed in claim 2, wherein:
the earbud has an elliptical shape when viewed axially, the elliptical shape having two relatively sharper ends and two relatively gentler sides;
the first emitter-to-sensor light transmission path arranged about one of the relatively sharper ends of the elliptical shape; and
the second emitter-to-sensor light transmission path arranged on one of the relatively gentler sides of the elliptical shape.

4. An ear-wearable physiology monitoring device as claimed in claim 2, wherein:
the earbud has an elliptical shape when viewed axially, the elliptical shape having two relatively sharper ends and two relatively gentler sides;
the first emitter-to-sensor light transmission path arranged about one of the relatively sharper ends of the elliptical shape; and
the second emitter-to-sensor light transmission path arranged about the other one of the relatively sharper ends of the elliptical shape.

5. An ear-wearable physiology monitoring device as claimed in claim 3, wherein:
the relatively sharper ends of the elliptical shape comprises a first end and a second end of the elliptical shape;
the first end of the elliptical shape being sharper than the second end of the elliptical shape;
the first end being capable of moving about the second end when the second end is adjacent the floor of an ear hole; wherein
the first emitter-to-sensor light transmission path is arranged about the second end.

6. An ear-wearable physiology monitoring device as claimed in claim 5, comprising:
an emitter and the at least two optical sensors providing the first emitter-to-sensor light transmission path and the second emitter-to-sensor light transmission path; wherein
the first emitter-to-sensor light transmission path comprises one of the at least two sensors.

7. An ear-wearable physiology monitoring device as claimed in claim 6, wherein:
the at least two sensors are placed in different locations along the axis of the earbud.

8. An ear-wearable physiology monitoring device as claimed in claim 5, comprising:
an optical sensor and the at least two emitters providing the first emitter-to-sensor light transmission path and the second emitter-to-sensor light transmission path; wherein
the first emitter-to-sensor light transmission path comprises one of the at least two emitters.

9. An ear-wearable physiology monitoring device as claimed in claim 8, comprising:
the at least two emitters are placed in different locations along the axis of the earbud.

10. A method for increasing diversity of movement artefacts in the signal noise of an ear-worn physiology monitoring device, comprising the steps of:
providing a first emitter-to-sensor light transmission path through ear tissue;
providing a second emitter-to-sensor light transmission path through ear tissue;
wherein the two different emitter-to-sensor light transmission paths have different extents of room for displacement in response to a movement of the user.

11. A method for increasing diversity of movement artefacts in the signal noise of an ear-worn physiology monitoring device as claimed in claim 10, further comprising the step of:
locating the first emitter-to-sensor light transmission path further from a point of rotation; and
locating the second emitter-to-sensor light transmission path nearer to the point of rotation; such that
in response to the user movement, the first emitter-to-sensor light transmission path is capable of moving about the point of rotation over a greater distance than the second emitter-to-sensor light transmission path according to the different extents of room for displacement.

12. A method for increasing diversity of movement artefacts in the signal noise of an ear-worn physiology monitoring device as claimed in claim 10, further comprising the step of:
performing a linear combination with a pre-defined ratio imposed on signals obtained from the two different emitter-to-sensor light transmission paths to remove movement artefacts.

13. An ear-wearable physiology monitoring device, comprising:
at least one emitter and at least one optical sensor in suitable numbers to provide a first emitter-to-sensor light transmission path through ear tissue and a second emitter-to-sensor light transmission path through ear tissue;
the first emitter-to-sensor light transmission path being spaced apart on the device from the second emitter-to-sensor light transmission path; wherein
the spacing provides that user movement causes a displacement of the first emitter-to-sensor light transmission path and a displacement of the second emitter-to-sensor light transmission path that is about a pivotal point distal from the axis of the ear hole.

14.-15. (canceled)
